(19) United States
(12) Patent Application Publication
(10) Pub. No.: US 2022/0400685 A1
(43) Pub. Date: Dec. 22, 2022

(54) CAMELLIA SINENSIS ENDOPHYTE PENICILLIUM EHRLICHII AND APPLICATION THEREOF

(71) Applicant: Anhui Agricultural University, Hefei (CN)

(72) Inventors: Hao JIANG, Hefei (CN); Li QIN, Hefei (CN); Hui WU, Hefei (CN); Xuehui LI, Hefei (CN); Mengting ZHANG, Hefei (CN)

(73) Assignee: Anhui Agricultural University, Hefei (CN)

(21) Appl. No.: 17/828,266

(22) Filed: May 31, 2022

(30) Foreign Application Priority Data
Jun. 21, 2021 (CN) 202110687884.2

(51) Int. Cl. A01N 63/36; C12N 1/14; A01P 3/00

(52) U.S. Cl. CPC A01N 63/36; C12N 1/145; A01P 3/00; C12R 2001/80

(57) ABSTRACT

The present disclosure belongs to the technical field of plant pest control, and particularly relates to a Camellia sinensis endophyte Penicillium ehrlichii and an application thereof. The present disclosure relates to a novel endophyte separated from a Camellia sinensis body and identified as Penicillium ehrlichii by means of morphology and molecular biology. Biological activity evaluation indicates that Penicillium ehrlichii shows good antagonistic antifungal activity to common pathogenic fungi on several plants. It means that the strain used as a biocontrol fungus for phytopathogens can be applied to environmental-friendly and safe prevention and control of plant diseases.

### CROSS-REFERENCE TO RELATED APPLICATIONS

The application claims priority to Chinese patent application No. 202110687884.2, filed on Jun. 21, 2021, the entire contents of which are incorporated herein by reference.

### TECHNICAL FIELD

The present disclosure belongs to the technical field of plant disease and pest control, and particularly relates to a Camellia sinensis endophyte Penicillium ehrlichii and an application thereof.

### BACKGROUND

Endophytes refer to microbial groups that are capable of living in intercellular spaces and cells of various organs and tissues of plants and establish a harmonious symbiotic relationship with plants. The microbial groups include bacteria, fungi, actinomycetes and the like. This harmonious symbiotic relationship shows that endophytes parasitize in host plants in their part or total life cycles and are not pathogenic to the plants temporarily. As one of components of the plants, endophytes play a significant role in functions of growth, biomass accumulation, carbon sink, biological remediation and the like of the plants.

Endophytes colonize in the host plants, which can promote plant growth on the one hand and can enhance resistance of the host plants to biotic stress (phytopathogens, injurious insects and the like) and abiotic stress on the other hand. Endophytes themselves generate a series of secondary metabolites or promote the host plants to generate a series of secondary metabolites in a certain development stage and under a certain external environment. Quite a number of secondary metabolites have very high biological activities and extensive application prospects. For example, they are applied to preparing antibiotics, immunosuppressors and the like. The biological control mechanism of endophytes includes antagonistic action (endophytes generate enzymes, antibiotics, bacteriocins and volatile substances) and induction of disease resistance of system (endophytes induce plants to generate polyphenol oxidase, peroxidase, phenylalnine ammonialyase and superoxide dismutase).

Plant diseases are one of major threats in production of agriculture and forestry. Chemical control is the major means for plant diseases for a long time, with increasingly prominent disadvantages such as drug resistance of pathogenic fungi, ecological environment pollution and threat to human health. Therefore, biological control has become one of important options capable of replacing chemical control. Besides, separation of biocontrol endophyte strains having the antagonistic action to pathogenic fungi are a premise and basis for development and utilization of biocontrol fungi for preventing and treating plant diseases.

Plant endophytes have various biocontrol mechanisms in biological control, and the biocontrol mechanisms of different endophytes are different. As biocontrol fungi, endophytes are different in mechanism of action in preventing and treating plant diseases. Endophytes primarily secrete antibiotic substances, compete with pathogens for nutrient substances and ecological niches, promote plant growth, induce plants to generate resistance systems and the like to inhibit growth of pathogenic fungi. At present, endophytes have been known as a natural resource for biological control. Due to their safety and high activities, endophytes are widely applied in environmental-friendly control of plant diseases and have wide application potential and development and application value.

Camellia sinensis, a perennial ligneous plant, is an important economic crop in China. Tea leaves are a natural healthy drink and are honored as a healthy beverage in the 21st century. However, plant diseases and insect pests that occur persistently will harm tea leave production severely. Traditional chemical control has hidden dangers in quality and health and safety of tea leaves, which has certain negative effects in tea industry. Control of plant diseases and insect pests in tea gardens by biopesticides (natural products such as microorganisms and plant sources) instead of chemical pesticides is of significance to environmental-friendly, healthy and sustainable development of tea industry. As a potential biological control resource, endophytes have a huge potential.

At present, it is late to research Camellia sinensis endophyte and there are few studies on Camellia sinensis endophyte. Meanwhile, there are few research reports on preventive effect of Camellia sinensis endophyte resources on Camellia sinensis diseases.

### SUMMARY

Aiming at problems in the prior art, the present disclosure provides a Camellia sinensis endophyte Penicillium ehrlichii and an application thereof to solve a part of problems in the prior art or at least relieve a part of problems in the prior art.

The present disclosure is realized in such a way that a Camellia sinensis endophyte Penicillium ehrlichii with preservation No. CCTCC NO: M 2021635 has been preserved in CCTCC on 31^{st}, May, 2021; Address: Wuhan University, Wuhan, China; Name of Culture: Penicillium ehrlichii Z19.

The present disclosure further provides an application of the Camellia sinensis endophyte Penicillium ehrlichii in preparation of a reagent in preventing and treating plant anthracnose.

Further, the plant anthracnose includes at least one of tea anthracnose, melon anthracnose, pear anthracnose and apple anthracnose.

The present disclosure further provides an application of the Camellia sinensis endophyte Penicillium ehrlichii in preparation of a reagent in preventing and treating leaf spot disease of plants.

Further, the leaf spot disease of plant includes at least one of tea leaf spot disease and Curvularia oryzae leaf spot disease.

The present disclosure further provides an application of the Camellia sinensis endophyte Penicillium ehrlichii in preparation of a reagent in preventing and treating plant wilt disease.

Further, the plant wilt disease includes at least one of cotton wilt disease and watermelon wilt disease.

The present disclosure further provides an application of the Camellia sinensis endophyte Penicillium ehrlichii in preparation of a reagent in preventing and treating grey seedling disease or Sclerotinia of plants or fruit disease of plants.

Further, the grey seedling disease of plants includes grey seedling disease of grapes, the Sclerotinia includes Sclerotinia rot of colza, and the fruit disease of the plants includes at least one of melon anthracnose, pear anthracnose and apple anthracnose.

The present disclosure further provides a biological control inoculant including the Camellia sinensis endophyte Penicillium ehrlichii.

The present disclosure relates to a novel endophyte separated from a Camellia sinensis body and identified as Penicillium ehrlichii by means of morphology and molecular biology. Biological activity evaluation indicates that Penicillium ehrlichii shows good antagonistic antifungal activity to common pathogenic fungi on several plants. It means that the strain used as a biocontrol fungus for phytopathogens can be applied to environmental-friendly and safe prevention and control of plant diseases. Of course, the present disclosure is not limited to several phytopathogens in the patent.

In conclusion, the present disclosure has following advantages and beneficial effects:

1. Biological Control

The Camellia sinensis endophyte Penicillium ehrlichii protected by the patent has very high antagonistic activity to several antagonistic activities. Compared with traditional chemical control, biocontrol fungi can prevent and treat plant diseases safely and efficiently without pollution, thereby reducing pollution to the ecological environment, improving the quality safety of agricultural products and guaranteeing the human health.

2. Endophyte as Biocontrol Strain

In the project, a novel Camellia sinensis endophyte Penicillium ehrlichii is separated from Camellia sinensis. At present, there have been no related reports on the strain used as biocontrol fungus and used for preventing and treating phytopathogens.

### BRIEF DESCRIPTION OF DRAWINGS

FIG. 1 shows inhibitory rate of an endophyte P. ehrlichii to 10 phytopathogens.

FIG. 2 shows an antifungal effect of an endophyte P. ehrlichii antagonistic to mycelial growth of an original pathogenic fungus of melon anthracnose for 7d.

FIG. 3 shows an antifungal effect of an endophyte P. ehrlichii antagonistic to mycelial growth of an original pathogenic fungus of tea zonate spot for 7d.

FIG. 4 shows an antifungal effect of an endophyte P. ehrlichii antagonistic to mycelial growth of an original pathogenic fungus of pear anthracnose for 7d.

FIG. 5 shows an antifungal effect of an endophyte P. ehrlichii antagonistic to mycelial growth of an original fungus of cotton wilt disease for 7d.

FIG. 6 shows an antifungal effect of an endophyte P. ehrlichii antagonistic to mycelial growth of an original pathogenic fungus of apple anthracnose for 7d.

FIG. 7 shows an antifungal effect of an endophyte P. ehrlichii antagonistic to mycelial growth of an original pathogenic fungus of grape grey mould for 7d.

FIG. 8 shows an antifungal effect of an endophyte P. ehrlichii antagonistic to mycelial growth of curvularia oryzae for 7 days.

FIG. 9 shows an antifungal effect of an endophyte P. ehrlichii antagonistic to mycelial growth of a pathogenic fungus of watermelon wilt disease for 7d.

FIG. 10 shows an antifungal effect of an endophyte P. ehrlichii antagonistic to mycelial growth of a pathogenic fungus of sclerotinia rot of colza for 7d.

FIG. 11 shows an antifungal effect of an endophyte P. ehrlichii antagonistic to mycelial growth of a pathogenic fungus of tea anthracnose for 7d.

### DETAILED DESCRIPTION OF THE EMBODIMENTS

In order to better understand the present disclosure rather than to limit the scope of the present disclosure, all numbers and other numerical values representing dosage and percent used in the present disclosure shall be understood as being modified by a word “approximate” under all circumstances. Therefore, unless otherwise specified, numerical parameters listed in the description and attached claims are approximate values which may be changed according to different ideal properties attempted to be obtained. All numerical parameters shall be regarded as being obtained by a conventional rounding method according to reported effective numbers. In the present disclosure, “approximate” refers to a set value or a value within 10% of a scope, preferably within 5% of a scope.

Unless otherwise defined, temperatures in the embodiments of the present disclosure are normal temperatures. Normal temperature refers to a natural room temperature condition in the four seasons without additional cooling or heating treatment. Normal temperature is generally controlled at 10-30° C., 15-25° C. the best.

The present disclosure discloses a Camellia sinensis endophyte Penicillium ehrlichii and an application thereof, specifically shown in the following embodiments.

### Example 1 Separation and Identification of an Endophyte Strain

Healthy Camellia sinensis root, stem and leaf tissues were taken, surface soil was cleaned with tap water, the healthy Camellia sinensis root, stem and leaf tissues were first immersed in 70% alcohol for 1 min, then placed in a 3% sodium hypochlorite solution to be immersed for 4 min, then placed in 70% alcohol to be rinsed for 1 s and finally washed with sterile water for 5 times, and 100 uL of the sterile water for last washing was absorbed and coated to a PDA culture medium to prepare a control culture medium.

A tissue separation method: a surface-disinfected Camellia sinensis tissue was sliced into pieces of 5 mm*5 mm, sections were attached to the PDA culture, the pieces were cultured at a constant temperature of 25° C. for 7d, observation was made every day, and new mycelia were picked out with a fungus moving ring and cultured independently.

ITS and 18S genetic locus sequencing was performed on the separated endophytic fungus, and BLAST comparison was made on the sequencing results on NCBI to obtain a similar strain named Penicillium ehrlichii (table 1).

TABLE 1
ITS sequencing identification result

| | | | | | | | Reference |
|---|---|---|---|---|---|---|---|
| Accession | Domain | Phylum | Class | Order | Family | Genus | species |
| NR_121229.1 | Fungi | Ascomycota | Eurotiomycetes | Eurotiales | Aspergillaceae | Penicillium | Penicillium |
| | | | | | | | ehrlichii |

### Example 2 an Antagonistic Experiment of Endophyte

In the example, plate antagonistic experiments were performed on the endophytic fungus P. ehrlichii against nine plant disease pathogenic fungi (melon anthracnose (Colletorichum lagenerium (Pass.) Ell. et Halst), tea zonate spot (Pseudopestalotiopsis theae), pear anthracnose (Colletrichum gloeosporioides Penz.), cotton wilt disease (Fusarium oxysporum), apple anthracnose (Colletotrichum gloeosporioides (Penz.) Penz. et Sacc.), grape grey mould (Botrytis cinerea Pers), curvularia oryzae leaf spot disease (Culvularia lunata), watermelon wilt disease (Fusarium oxysporum), and sclerotinia rot of colza (Sclerotinia sclerotiorum (Lib.) de Bary)), diameters of growing mycelia were counted at different culture times, and the inhibitory rates were calculated. The specific contents are as follows:

1. Plate Antagonistic Method:

A fungal cake was cut from the edge of a fungal colony of a pathogenic fungus and inoculated to a center point of a plate of the PDA culture medium with mycelia facing downwards, four endophyte P. ehrlichii fungal cakes were cut by the same method and inoculated to four directions that were 2.5 cm from the fungal cake of the pathogenic fungus respectively, a plate without P. ehrlichii fungal cakes was used as control, the fungal cake was cultured at a constant temperature of 25° C. for 7d, the diameter of the fungal colony of the pathogenic fungus was measured every day, and the inhibitory rate was calculated. A calculating method for inhibitory rate:

The inhibitory rate of an antagonistic fungus (%)=((the diameter of the fungal colony in the control group-5)−(the diameter of the fungal colony in the treatment group-5))/(the diameter of the fungal colony in the control group-5)

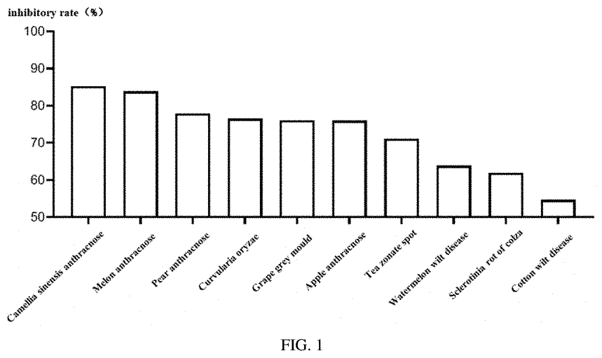

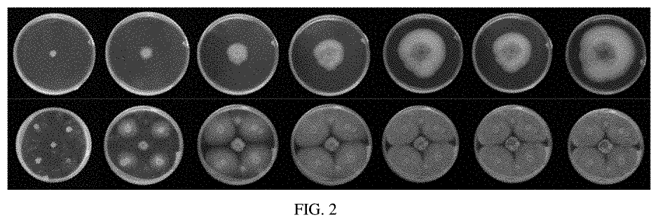

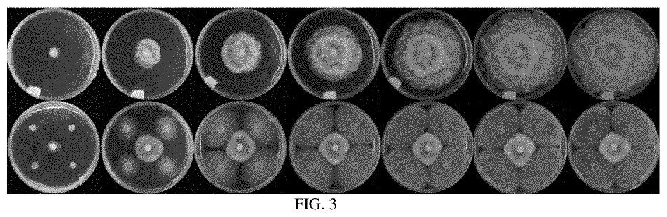

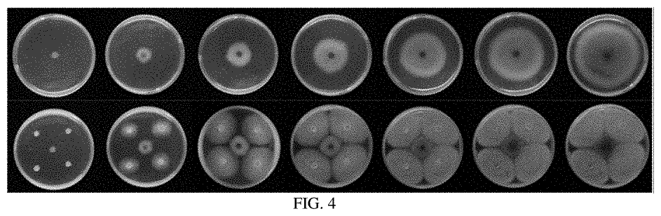

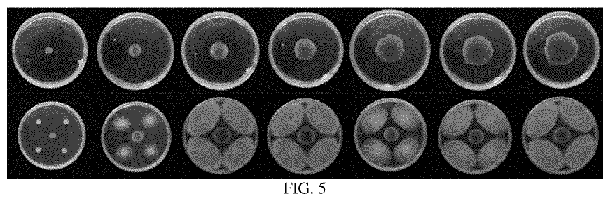

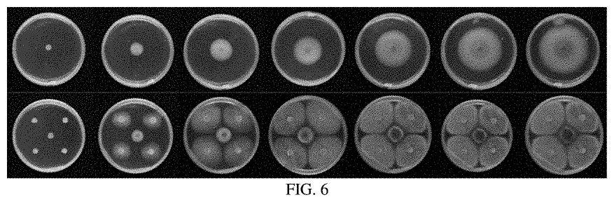

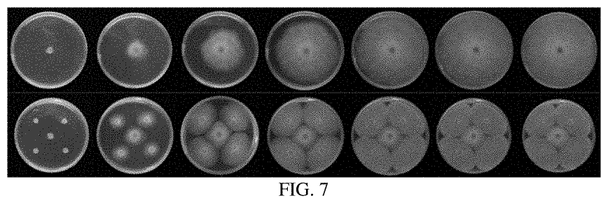

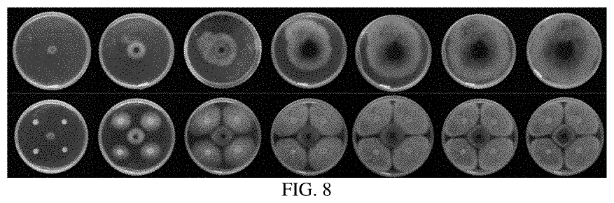

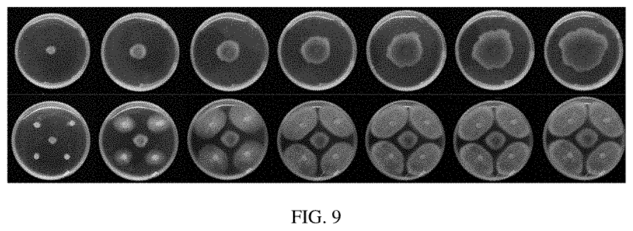

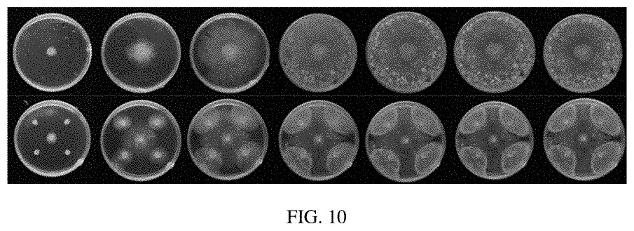

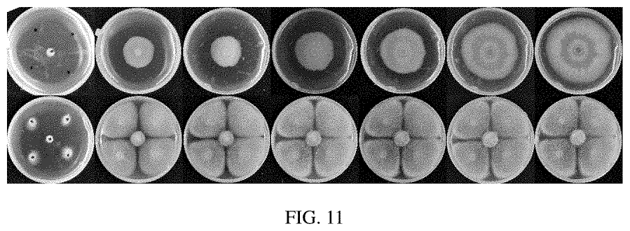

2. An antagonistic experiment result: researches showed that on the seventh day, the inhibitory rates of P. ehrlichii to the selected 10 pathogenic fungi were 50% or above (FIG. 1), nearly 80% or above for prevention and treatment of anthracnose. Effect diagrams of the endophytic fungus P. ehrlichii to growth inhibition effect of mycelia of 10 phytopathogens were shown in FIG. 2-11. The effect diagrams of the control group were on the upper side and the effect diagrams of the treatment group were on the lower side.

The above mentioned is merely the preferred embodiment of the present disclosure and is not used to limit the present disclosure. Any modification, equivalent replacement, improvement, etc. made within the spirit and principle of the present disclosure shall be regarded as within the protection scope of the present disclosure.

1. A Camellia sinensis endophyte Penicillium ehrlichii, with a preservation number: CCTCC NO: M 2021635.

2. An application of the Camellia sinensis endophyte Penicillium ehrlichii according to claim 1 in preparation of a reagent in preventing and treating plant anthracnose wherein the plant anthracnose comprises at least one of tea anthracnose, melon anthracnose, pear anthracnose and apple anthracnose.

3. (canceled)

4. An application of the Camellia sinensis endophyte Penicillium ehrlichii according to claim 1 in preparation of a reagent in preventing and treating leaf spot disease of plants, wherein the leaf spot disease of plant comprises at least one of tea leaf spot disease and Curvularia oryzae leaf spot disease.

5. (canceled)

6. An application of the Camellia sinensis endophyte Penicillium ehrlichii according to claim 1 in preparation of a reagent in preventing and treating plant wilt disease wherein the plant wilt disease comprises at least one of cotton wilt disease and watermelon wilt disease.

7. (canceled)

8. An application of the Camellia sinensis endophyte Penicillium ehrlichii according to claim 1 in preparation of a reagent in preventing and treating grey seedling disease or Sclerotinia of plants or fruit disease of plants, wherein the grey seedling disease of plants comprises grey seedling disease of grapes, the Sclerotinia includes Sclerotinia rot of colza, and the fruit disease of the plants includes at least one of melon anthracnose, pear anthracnose and apple anthracnose.

9. (canceled)

10. A biological control inoculant, comprising the Camellia sinensis endophyte Penicillium ehrlichii according to claim 1.
